(19) United States
(12) Patent Application Publication
(10) Pub. No.: US 2023/0174908 A1
(43) Pub. Date: Jun. 8, 2023

(54) REAGENT INJECTIONS INTO CELLS

(71) Applicant: HEWLETT-PACKARD DEVELOPMENT COMPANY, L.P., Spring, TX (US)

(72) Inventors: Alexander GOVYADINOV, Corvallis, OR (US); Viktor SHKOLNIKOV, Palo Alto, CA (US)

(73) Assignee: HEWLETT-PACKARD DEVELOPMENT COMPANY, L.P., Spring, TX (US)

(21) Appl. No.: 17/911,553

(22) PCT Filed: Apr. 30, 2020
(86) PCT No.: PCT/US2020/030715

§ 371 Date: Sep. 14, 2022

(51) Int. Cl. C12M 3/00; C12M 1/42; C12M 1/00

(52) U.S. Cl. CPC C12M 3/006; C12M 35/08; C12M 31/00

(57) ABSTRACT

In example implementations, an apparatus is provided. The apparatus includes a channel, a reagent chamber, a synthetic jet channel, and an energy source. The channel is to hold a cell. The reagent chamber is coupled to the channel and stores a reagent. The synthetic jet channel is coupled to the channel and the reagent chamber. The energy source is located in the synthetic jet channel to heat a liquid in the synthetic jet channel to create a synthetic jet that carries the reagent through towards the cell to inject the reagent into the cell.

### BACKGROUND

Cell transfection may be used for research and production of certain biological products, such as synthetic proteins, genetically modified organisms, and the like. Cell transfection includes creating pores in a cell membrane of a cell and inserting a foreign material into the cell.

### BRIEF DESCRIPTION OF THE DRAWINGS

FIG. 1 is a block diagram of an example system to inject reagents into cells of the present disclosure;

FIG. 2 is a block diagram of a cross-sectional view of an example apparatus with an energy source to generate a synthetic jet to inject a reagent into a cell of the present disclosure;

FIG. 3 is a block diagram of a top view of the example apparatus with a plurality of different reagent chambers and energy sources of the present disclosure;

FIG. 4 is a block diagram of a cross-sectional view of the example apparatus with cell trapping, multiple energy sources, and a pump of the present disclosure;

FIG. 5 is a block diagram of a cross-sectional view an example apparatus having a sensor and a feedback loop of the present disclosure;

FIG. 6 is a block diagram of a cross-sectional view an example apparatus with nested reagent supply chambers of the present disclosure;

FIG. 7 is a block diagram of a cross-sectional view an example apparatus with a series of reagent injections;

FIG. 8 is a block diagram of a cross-sectional view of an example apparatus with multiple layers; and

FIG. 9 is a flow chart of an example method for transfection injections of the present disclosure.

### DETAILED DESCRIPTION

Examples described herein provide a system and apparatus for reagent injections into a cell. As noted above, transfection of cells can be used for research or production of certain biological products. Transfection allows the behavior of the cell to be changed. For example, by diffusing a reagent, such as a particular DNA along with proteins that incorporate the DNA into a cell's genome, the cell's genome may be altered to create a genetically modified organism.

Some systems to perform the poration and transfection can potentially introduce contamination. Some methods for poration use expensive tooling or may not provide sufficient control of the amount of material that is transfected into a cell. Other methods may also not allow for transfection with microscopic amounts of fluids in single cells.

Examples herein provide a system and apparatus for direct injection of reagents into a cell. The apparatus may use an energy source, such as a thermal inkjet (TIJ) resistor that can generate a synthetic jet. For example, the energy source may create local vapor bubbles that may burst to create the synthetic jet in a direction towards the cell. The synthetic jet may carry a desired reagent at a velocity that can inject the reagent into the cell, thereby performing a transfection injection.

In one example, the system may also provide a sensor and a feedback control loop. For example, sensors may be implemented to detect the presence of a cell to control operation of the energy source. As a result, the energy source may be activated and deactivated based on whether cells are present in the apparatus. The feedback loop may determine if the injection of the reagent was successful and move the cell to its destination or return the cell for further injections of reagents based on the feedback loop.

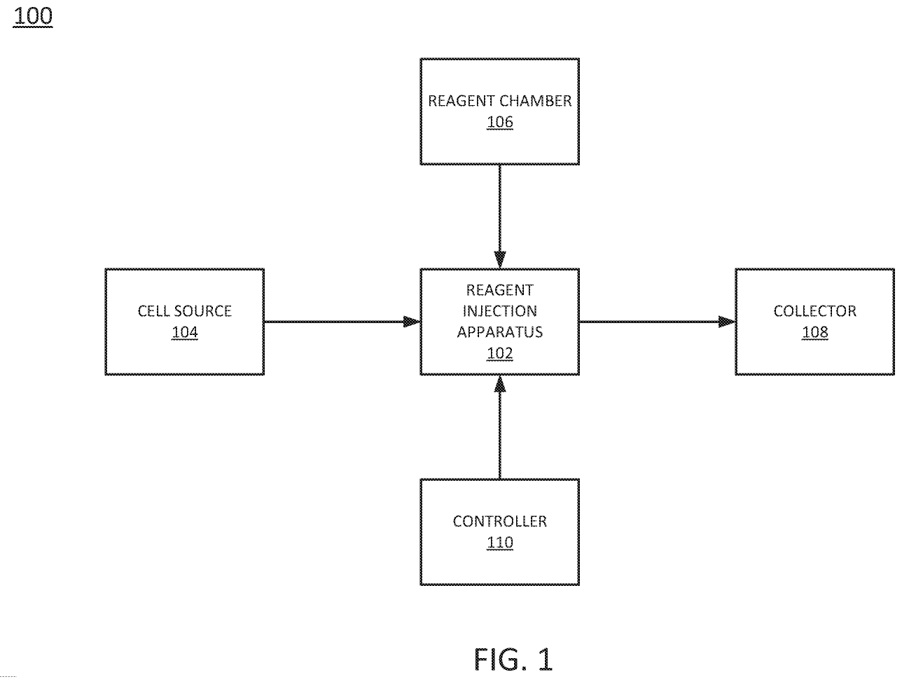

FIG. 1 illustrates an example block diagram of a system 100 of the present disclosure. In an example, the system 100 may include a reagent injection apparatus 102, a cell source 104, a reagent chamber 106, a collector 108, and a controller 110. Although illustrated as separate components, in an example, the reagent injection apparatus 102, the cell source 104, the reagent chamber 106, the collector 108, and the controller 110 may be combined into a single housing or device.

In an example, the controller 110 may be a processor or application specific integrated chip (ASIC) that can be programmed to perform certain functions. The controller 110 may be communicatively coupled to various devices in the reagent injection apparatus 102, as discussed in further details below, to control operation of the reagent injection apparatus 102.

In an example, cells from the cell source 104 may be fed through the reagent injection apparatus 102. The reagent injection apparatus 102 may inject a reagent from the reagent chamber 106 into the cells. As discussed in further details below, the reagent chamber 106 may include a single chamber or multiple chambers that include different types of reagents. The multiple chambers may be different chambers that inject reagents into a cell in a serial fashion, or the reagents may be fed into a mixing chamber and mixed to inject the cell with a mixture of reagents.

After a cell is injected with a reagent or reagents, the cell may be fed to the collector 108. The collector 108 may be a collection apparatus such as a container, individual wells of a well array, and the like. In an example, the collector 108 may form a continuous loop. For example, the cells may travel from the collector 108 back to the cell source 104. For example, some cells may not be injected when fed through the reagent injection apparatus 102. As a result, some cells may be collected in the collector 108 and other cells may be fed back to the cell source 104 to form a loop.

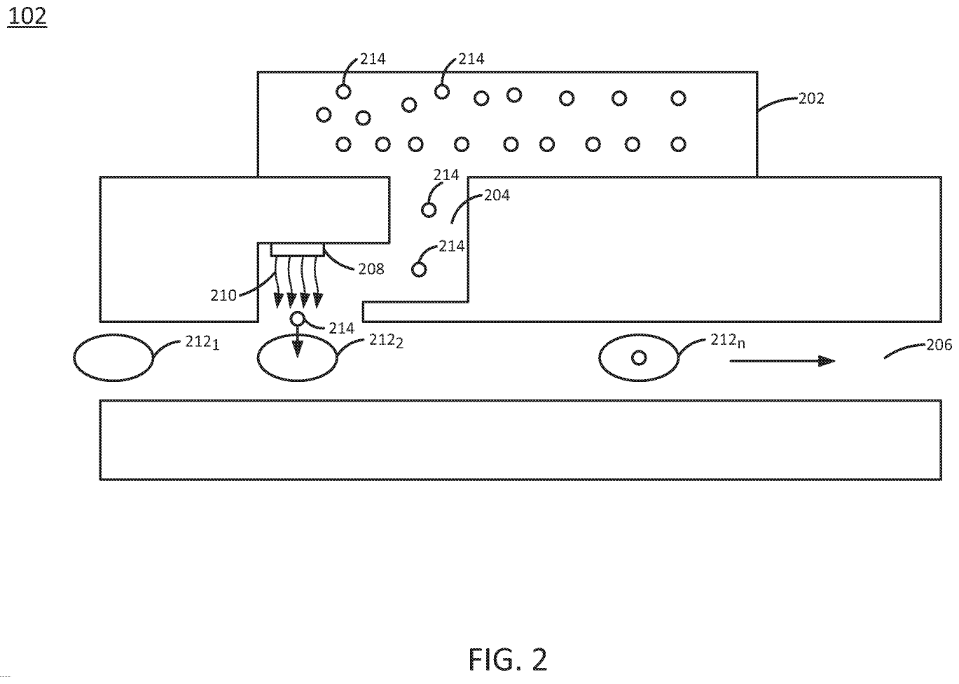

FIG. 2 illustrates a cross-sectional block diagram of an example of the reagent injection apparatus 102 of the present disclosure. In an example, the reagent injection apparatus 102 may include a reagent chamber 202, a synthetic jet channel 204, and a channel 206. Cells 212₁ to 212ₙ (hereinafter also referred to individually as a cell 212 or collectively as cells 212) are fed towards the synthetic jet channel 204 through the channel 206. In an example, the cells 212 may be moved through the channel 206 via a pump (not shown), gravity, capillary force, centrifugal force, electrophoretic force, dielectrophoretic force, and the like.

In an example, the sizing of the channel 206 may be a function of the size of the cells 212. The sizing may refer to the dimensions (e.g., length and width) of the cross-sectional opening of the channel 206. The size of the channel 206 may be large enough to allow the passage of a single cell 212 at a time.

In example, the synthetic jet channel 204 may be a volume located between an opening of the reagent chamber 202 and an opening in the channel 206. The synthetic jet channel 204 may include an energy source 208 to heat a liquid in the synthetic jet channel 204 to generate a synthetic jet 210. The synthetic jet 210 may move at a velocity sufficient to carry a reagent 214 in the synthetic jet channel 204 towards the cells 212. For example, a cell 212₂ may be positioned adjacent to the synthetic jet channel 204 and the energy source 208 to receive the reagent 214. The synthetic jet 210 may porate the cell 212₂ to allow the reagent 214 to be injected into the cell 212₂. Thus, when the term “injection” is used herein, it may include the action of poration of the cell and insertion of the reagent 214. The synthetic jet 210 may also move the reagent 214 towards the cell 212₂ to inject the reagent 214 into the cell 212₂.

In an example, the synthetic jet 210 may be defined as a jet created within the liquid in the synthetic jet channel 204. In other words, the synthetic jet 210 is formed from the surrounding liquid rather than an external source or fluid. In an example, the liquid may be water, a solvent, or any other liquid that is compatible with the reagent 214 and the cells 212.

In an example, the synthetic jet 210 may be formed by the energy source 208. The energy source 208 may locally heat the liquid in the synthetic jet channel 204 to create vapor bubbles of the liquid. As the vapor bubbles burst, the energy released by the bursting vapor bubbles may create the synthetic jet 210. The synthetic jet 210 may move in a direction of the bursting vapor bubbles. The energy source 208 may be arranged to direct the synthetic jet 210 towards an opening of the synthetic jet channel 204 where a cell 212 is located to receive the reagent 214.

In an example, the energy source 208 may be an inductive heater or a resistor heater. An example of a resistor heater may be a thermal inkjet (TIJ) resistor. A TIJ resistor may include a controllable circuit that includes a resistor heater. When the circuit is activated, current may flow through the resistor heater to generate heat.

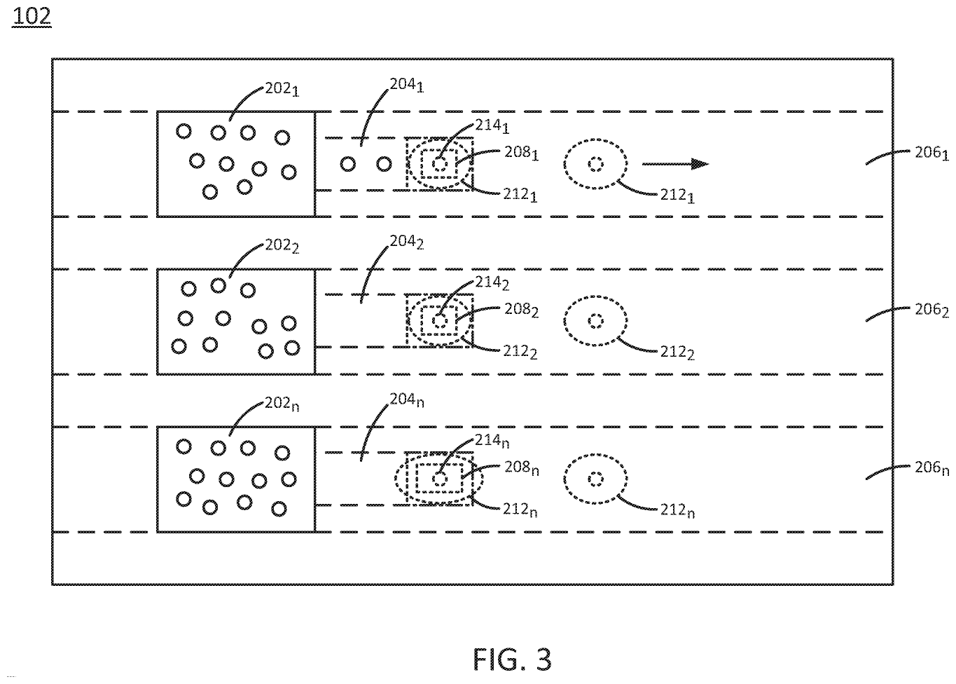

FIG. 3 illustrates a block diagram of a top view of the example reagent injection apparatus 102. The top view illustrates how the reagent injection apparatus 102 may include a plurality of channels 206₁ to 206ₙ (hereinafter also referred to individually as a channel 206 or collectively as channels 206). Each channel 206 can include a respective synthetic jet channel 204 and a respective energy source 208.

Each channel 206 may include a respective reagent chamber 202₁ to 202ₙ (hereinafter also referred to individually as a reagent chamber 202 or collectively as reagent chambers 202). The reagent chambers 202 may store a reagent 214. As discussed in examples below, the reagent chambers 202 may include different types of reagents 214 or the same reagent 214.

The cells 212 may be fed through the reagent injection apparatus 102. The cells 212 may flow through any of the channels 206₁ to 206ₙ. In another example, different cell sources 104 may be coupled to each channel 206 to control which cells 212 flow through which channels 206.

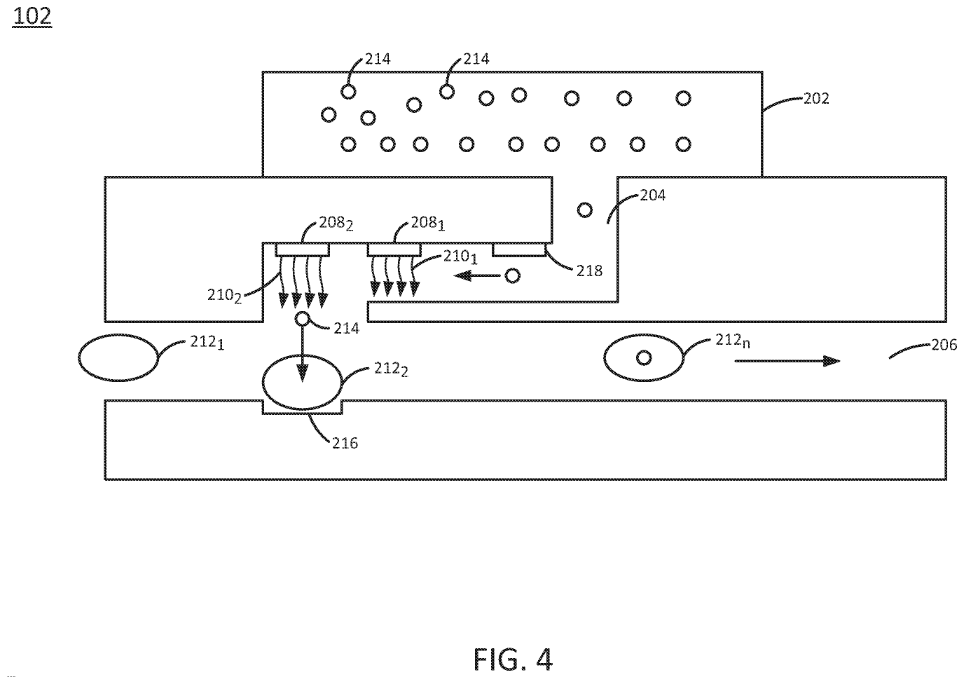

In an example, the channels 206 may be a single large channel with structures to guide the cells 212 towards the energy source 208. For example, poles, chevrons, or other physical guides, may move the cells 212 in a single channel to various openings of synthetic jet channels 204 and energy sources 208. In an example, cell trapping features (described below and illustrated in FIG. 4) may be used to temporarily position the cells 212 to receive the reagent 214.

In an example, the reagent 214 may be injected into the cells 212 when the cells 212 are positioned below the energy source 208. The energy source 208 may generate the synthetic jet 210 to carry the reagent 214 towards the cells 212. The reagent 214 may be injected into the cells 212 via the force of the synthetic jet 210.

Although FIG. 3 illustrates the channels 206 having some symmetry, it should be noted that the channels 206 may be asymmetric. In other words, the reagent chambers 202, the synthetic jet channels 204, and the energy sources 208 may be positioned differently in each channel 206. In addition, the channels 206 may be arranged with even or uneven spacing between the channels 206.

FIG. 4 illustrates a block diagram of a cross-sectional view of the example reagent injection apparatus 102 with a cell trapping feature 216, multiple energy sources 208, and a pump 218. It should be noted that any of the features may be included, removed, or combined within the example reagent injection apparatus 102.

In an example, the pump 218 may be located near an opening adjacent to the reagent chamber 202. The pump 218 may help the reagent 214 to flow towards the energy sources 208₁ and 208₂ in the synthetic jet channel 204. The pump 218 may be a microfluidic pump that comprises an electrode or heater. The pump 218 may generate flow towards the energy sources 208₁ and 208₂ via vapor bubbles generated by heating the liquid, pulses in the liquid, vibrations in the liquid, flow generated by temperature differentials in the liquid, and the like.

In an example, the synthetic jet channel 204 may include a plurality of energy sources 208₁ and 208₂. The energy sources 208₁ and 208₂ may be arranged in series within the synthetic channel 204 or arranged in parallel. Although two energy sources 208₁ and 208₂ are illustrated in FIG. 4, it should be noted that any number of energy sources 208 may be deployed.

In an example, each energy source 208₁ and 208₂ may generate a respective synthetic jet 210₁ and 210₂. The combined effect of the synthetic jets 210₁ and 210₂ may be to move the reagent 214 with greater velocity and force than using a single energy source 208. Thus, in certain applications where more force is used to inject the reagent 214 into the cell 212, multiple energy sources 208₁ and 208₂ may be deployed.

In an example, the number of energy sources 208₁ and 208₂ may be a function of a desired velocity of the reagent 214 to inject into the cell 212. For example, the effect of each synthetic jet 210 generated by an energy source 208 on the reagent 214 within known dimensions of the synthetic jet channel 204 may be known. The velocity at which the reagent 214 should be moving to be injected into the cell 212 may be known. Based on this information, the number of energy sources 208 that should be deployed in synthetic jet channel 204 may be calculated.

In an example, the reagent injection apparatus 102 may also include a cell trapping feature 216. The cell trapping feature 216 may temporarily hold the cell 212 in position to receive the reagent 214. In example, the position may be adjacent to and/or below an opening of the synthetic jet channel 204 where the reagent 214 exits. In an example, the position may be below an energy source 208 located in the synthetic jet channel 204.

In an example, the cell trapping feature 216 may be a mechanical feature. For example, the cell trapping feature 216 may a cut-out or notch formed in the channel 206. FIG. 4 illustrates an example where the cell 212₂ may drop or get caught by the cut-out. The cell 212₂ may be positioned to receive the reagent 214. The force of a collision from a subsequent cell 212₁ moving through the channel 206 may move the cell 212₂ out of the cut-out. The cell 212₁ may then be trapped in the cut-out to receive the reagent 214.

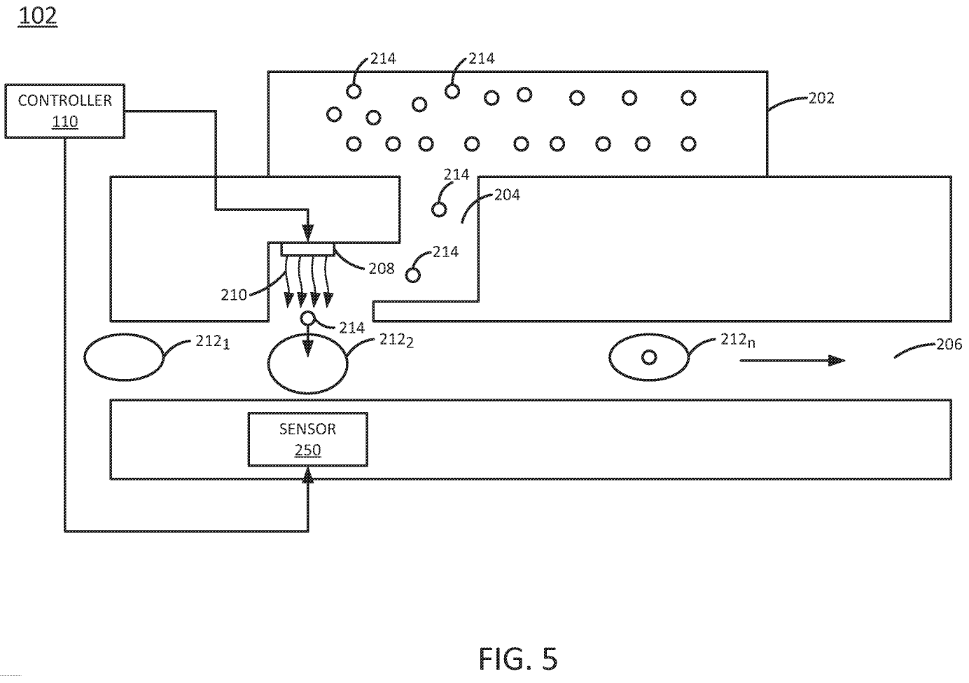

FIG. 5 illustrates a block diagram of a cross-sectional view the example reagent injection apparatus 102 with a sensor and a feedback loop. In an example, the reagent injection apparatus 102 may include a feedback loop to detect the presence of a cell 212 in a desired position to receive the reagent 214 and/or detect that the reagent 214 has been successfully injected into the cell 212.

In an example, the feedback loop may include a sensor 250. The sensor 250 may be communicatively coupled to the controller 110. The controller 110 may also be communicatively coupled to the energy source 208. The controller 110 may activate the energy source 208 based on a signal received from the sensor 250.

For example, when the cell 212₂ is detected by the sensor 250, the sensor 250 may send a first signal to the controller 110 indicating detection of the cell 212₂. In response to the detection signal, the controller 110 may activate the energy source 208. The energy source 208 may create the synthetic jet 210 to carry the reagent 214 towards the cell 212₂ to inject the reagent 214 into the cell 212₂. When the cell 212₂ moves down the channel 206 away from the sensor 250, the sensor 250 may send a second signal to the controller 110. In response to the second signal, the controller 110 may deactivate the energy source 208. The controller 110 may control each energy source 208 in each synthetic jet channel 204 of the reagent injection apparatus 102 similarly.

In an example, the sensor 250 may be an optical sensor or an electronic sensor. For example, the sensor 250 may include a collection optic (e.g., a red, green, blue (RGB) camera), a photosensor, an imaging array, or another type of video capturing device. A light source may apply a light to the channel 206 where the RGB camera captures images. When the RGB camera captures an image that includes the cell 212₂, a signal may be sent to the controller 110.

In an example, the sensor 250 may be an impedance sensor or an electrode that detects the presence of the cell 212₂. For example, the resistance of the impedance sensor may change when the cell 212₂ touches the sensor. In response, the impedance sensor may send a signal to the controller 110.

In an example, the controller 110 can control the energy source 208 based on signals from the sensor 250. For example, the controller 110 may keep the energy source 208 activated while the sensor 250 detects the cell 212₂. When the sensor 250 does not detect the cell 212₂, the controller 110 may deactivate the energy source 208. For example, the sensor 208 may send a non-detection signal to the controller 110. The non-detection signal may also be an absence of a signal from the sensor 208 (e.g., the sensor 208 stops sending a detection signal to the controller 110).

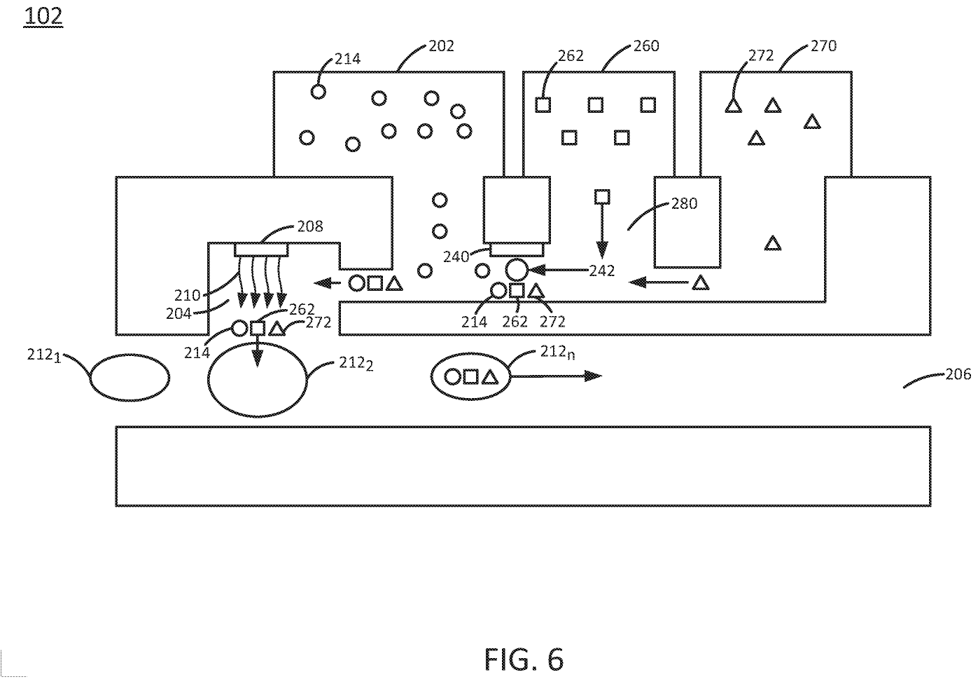

FIG. 6 illustrates a block diagram of a cross-sectional view of the example reagent injection apparatus 102 with nested reagent supply chambers. In an example, the reagent injection apparatus 102 may include a plurality of reagent chambers 202, 260, and 270. Although three reagent chambers are illustrated in FIG. 6, it should be noted that any number of reagent chambers can be deployed.

The reagent chambers 202, 260, and 270 may store different reagents 214, 262, and 272, respectively. In an example, the cells 212 may be injected with the reagents 214, 262, and 272 in a single injection through the synthetic jet channel 204.

In an example, the reagent injection apparatus 102 may include a reagent mixing chamber 280. The reagent mixing chamber 280 may include an energy source 240 to mix the reagents 214, 262, and 272. For example, the reagents 214, 262, and 272 may be fed into the reagent mixing chamber 280 from the respective reagent chambers 202, 260, and 270, respectively. The energy source 240 may be an inductive heater or a resistor heater (e.g., a TIJ resistor). The energy source 240 may be activated to mix the reagents 214, 262, and 272.

The mixture of the reagents 214, 262, and 272 may be fed towards the synthetic jet channel 204. When the cell 212₂ is in position to receive the mixture of reagents 214, 262, and 272, the energy source 208 may be activated to generate the synthetic jet 210. The synthetic jet 210 may carry the mixture of reagents 214, 262, and 272 towards the cell 212₂ to inject the cell with the mixture of reagents 214, 262, and 272.

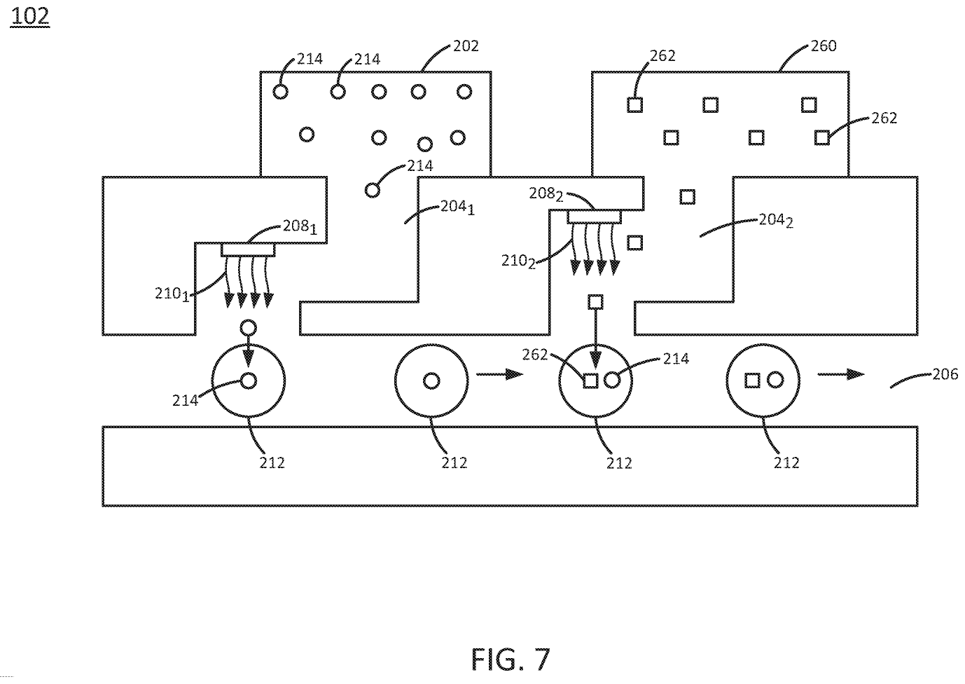

FIG. 7 illustrates a block diagram of a cross-sectional view of the example reagent injection apparatus 102 with a series of reagent injections. In an example, the reagent injection apparatus 102 may include a plurality of reagent chambers 202 and 260 arranged in series. Although two reagent chambers are illustrated in FIG. 7, it should be noted that any number of reagent chambers can be deployed.

The reagent chambers 202 and 260 may store different reagents 214 and 262, respectively. In an example, the cell 212 may travel down the channel 206 and be injected with the reagents 214 and 262 in series. For example, the cell 212 may be positioned below the synthetic jet channel 204₁ to be injected with the reagent 214. For example, the energy source 208₁ may be activated to generate the synthetic jet 210₁. The synthetic jet 210₁ may inject the cell 212 with the reagent 214.

After the reagent 214 is injected in to the cell 212, the cell 212 may be moved down the channel 206 to the synthetic jet channel 204₂. The synthetic jet channel 204₂, may inject the reagent 262 into the cell 212 via the energy source 208₂. For example, the energy source 208₂ may be activated to generate the synthetic jet 210₂. The synthetic jet 210₂ may inject the cell 212 with the reagent 262.

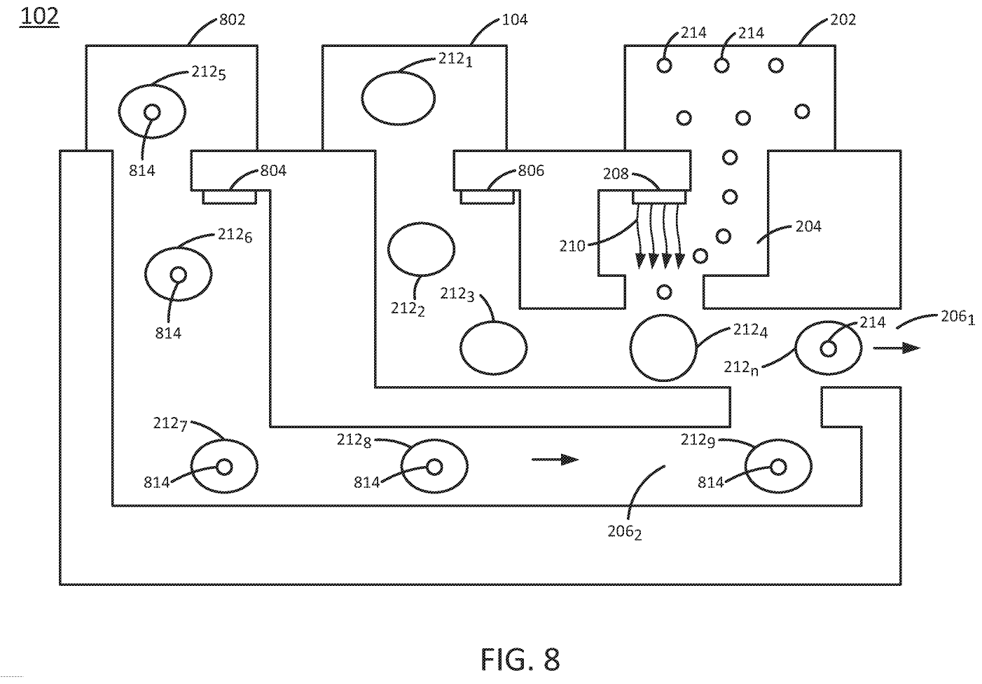

FIG. 8 illustrates a block diagram of a cross-sectional view of the reagent injection apparatus 102 that includes multiple layers or channels. In an example, the reagent injection apparatus 102 may include multiple layers of channels 206₁ and 206₂. The reagent injection apparatus 102 may include a cell source 802, a cell source 104, and a reagent chamber 202.

In one example, the reagent chamber 202 may include reagents 214 that are fed through a synthetic jet channel 204 and injected into the cells 212 via the energy source 208. As described above, the energy source 208 may generate the synthetic jet 210 that injects the reagent 214 into the cell 212.

In an example, the reagent injection apparatus 102 may include cells 212₁-212ₙ. Some of the cells 212₁ to 212₄ may be fed from the cell source 104 via a pump 806 (e.g., similar to the pump 240 illustrated in FIG. 6 and described above) and injected with the reagent 214 in the channel 206₁. Other cells 212₅-212₉ may be fed from the cell source 802 via a pump 804 (e.g., similar to the pump 240 illustrated in FIG. 6 and described above) through the channel 206₂.

In an example, the cell source 802 may include cells 212₅-212₉ that are injected with a reagent 814 from a previous injection operation within another portion of the reagent injection apparatus 102 or collected from another external source. The reagent 814 may be the same as the reagent 214 or may be a different reagent. The cells 212₅-212₉ may be fed through the channel 206₂ and combined with the cells 212₁ to 212₄ in the channel 206₁. The cells 212₁-212ₙ can then be sent for further collection, sorting, handling, and the like.

It should be noted that although two channels 206₁ and 206₂ are illustrated in FIG. 8 that any number of channels or layers may be deployed in the reagent injection apparatus 102. For example, different cells may be injected in different channels at different layers and then combined.

It should be noted that various examples of the reagent injection apparatus 102 are illustrated and described above. Although various features are illustrated in different figures, it should be noted that each figure is shown to illustrate various example features. In other words, features shown one figure may be mixed or combined with features in the other figures. For example, the cell trapping feature 216 illustrated in FIG. 4 may deployed with the sensor 250 illustrated in FIG. 5. The sensor 250 illustrated in FIG. 5 may be deployed with the multiple reagent chamber examples illustrated in FIGS. 6 and 7. The pump 218 illustrated in FIG. 4, may be used in the reagent mixing chamber 280 with the energy source 240 illustrated in FIG. 6, and so forth.

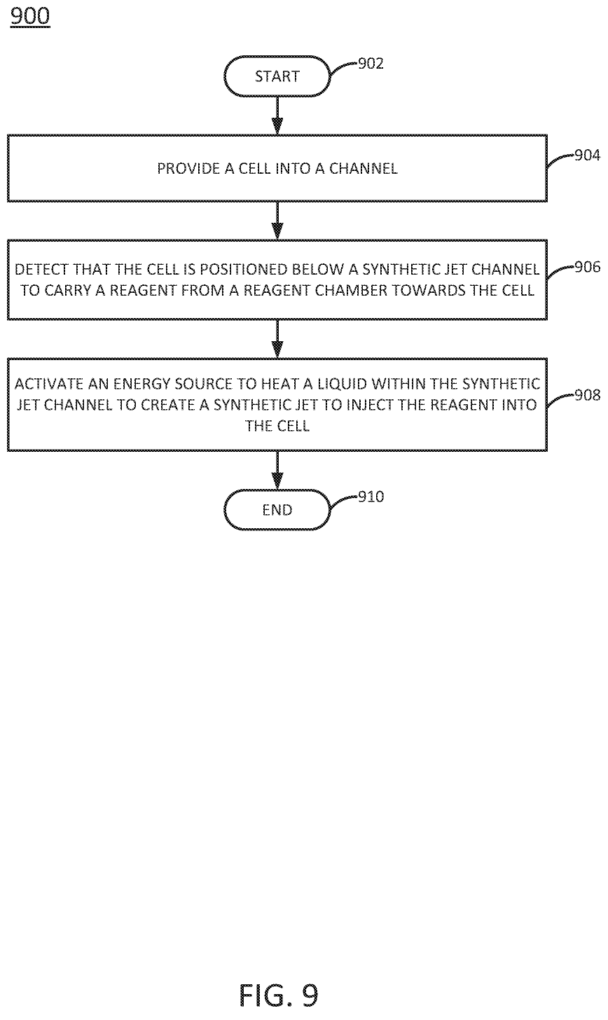

FIG. 9 illustrates a flow diagram of an example method 900 for injecting reagents into a cell of the present disclosure. In an example, the method 900 may be performed by the apparatus 102.

At block 902, the method 900 begins. At block 904, the method 900 provides a cell into a channel. For example, a cell may be fed from a cell source towards a channel of a reagent injection apparatus.

At block 906, the method 900 detects that the cell is positioned below a synthetic jet channel to carry a reagent from a reagent chamber towards the cell. In an example, a cell trapping feature can be used to position the cell below the synthetic jet channel. In an example, a sensor (e.g., optical or electrical sensor) may be used to detect that the cell is in a correct position below the synthetic jet channel.

At block 908, the method 900 activates an energy source to heat a liquid within the synthetic jet channel to create a synthetic jet to inject the reagent into the cell. For example, the energy source may be an inductive heater or a resistor heater (e.g., a TIJ resistor). The energy source may create vapor bubbles in a liquid inside of the synthetic jet channel. The vapor bubbles may burst to release energy creating a synthetic jet within the liquid in the synthetic jet channel.

The synthetic jet may move with enough force to porate the cell. The synthetic jet may also carry the reagent towards the cell such that the reagent is carried into the cell through the porations formed on the cell. Thus, the synthetic jet may inject the reagent into the cell.

In an example, the blocks 906 and 908 may be repeated for multiple synthetic jet channels where multiple reagents may be injected into the cell in series. For example, different reagents may be injected into the cell via different respective synthetic jet channels, as shown in FIG. 7 and discussed above.

In an example, the cells may be transported to an appropriate collector. For example, if the reagent is successfully injected into the cell, the cell may be directed towards a collector (e.g., a storage container, a particular well of a well array, and the like). However, if the reagent failed to be injected into the cell, the cell may be recycled back to the cell source. The cell may then be fed again through the reagent injection apparatus to attempt to inject the reagent into the cell again. At block 910, the method 900 ends.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

1. An apparatus, comprising:
a channel to hold a cell;
a reagent chamber coupled to the channel to store a reagent;
a synthetic jet channel coupled to the channel and the reagent chamber; and
an energy source located in the synthetic jet channel to heat a liquid in the synthetic jet channel to create a synthetic jet that carries the reagent towards the cell to inject the reagent into the cell.

2. The apparatus of claim 1, wherein the energy source comprises a thermal inkjet resistor.

3. The apparatus of claim 1, wherein the channel comprises an indentation to hold the cell in a position that is aligned with the energy source.

4. The apparatus of claim 1, further comprising:
a plurality of reagent chambers, wherein each one of the plurality of reagent chambers stores a different reagent to be injected into the cell; and
a plurality of synthetic jet channels, wherein each one of the plurality of synthetic jet channels includes a respective energy source to generate a respective synthetic jet.

5. The apparatus of claim 1, wherein the reagent chamber includes a plurality of different reagents to be mixed before being injected into the cell.

6. The apparatus of claim 1, further comprising:
a pump inside of the reagent chamber to move the reagent towards the synthetic jet channel and the energy source to be injected into the cell.

7. The apparatus of claim 1, further comprising:
a second reagent chamber coupled to store a second reagent;
a second synthetic jet channel coupled to the second reagent chamber and the channel; and
a second energy source located in the second synthetic jet channel to generate a second synthetic jet that carries the second reagent towards a second cell held in the channel to inject the second reagent into the second cell, wherein the reagent and the second reagent are injected simultaneously in the channel.

8. An apparatus, comprising:
a channel to hold a cell;
a reagent chamber coupled to the channel to store a reagent;
a synthetic jet channel coupled to the channel and the reagent chamber;
an energy source located in the synthetic jet channel to heat a liquid within the synthetic jet channel to create a synthetic jet to inject the reagent into the cell;
a sensor located upstream from the energy source; and
a controller communicatively coupled to the sensor, wherein the controller is to:
detect the presence of the cell in the channel based on a signal received from the sensor; and
activate the energy source to generate the synthetic jet and to inject the reagent into the cell in response to a detection of the cell in the channel.

9. The apparatus of claim 8, wherein the sensor comprises an impedance sensor or a capacitive sensor.

10. The apparatus of claim 8, wherein the controller is to deactivate the energy source in response to a non-detection signal from the sensor.

11. The apparatus of claim 10, wherein the sensor comprises a photosensor.

12. The apparatus of claim 10, wherein the sensor comprises:
an illumination source; and
a collection optic to capture a plurality of images of the cell to detect the reagent inside of the cell.

13. A method, comprising:
providing a cell into a channel;
detecting that the cell is positioned below a synthetic jet channel to carry a reagent from a reagent chamber towards the cell; and
activating an energy source to heat a liquid within the synthetic jet channel to create a synthetic jet to inject the reagent into the cell.

14. The method of claim 13, further comprising:
moving the cell downstream in the channel;
detecting that the cell is positioned below a second synthetic jet channel to carry a second reagent from a second reagent chamber towards the cell; and
activating a second energy source to generate a second synthetic jet to inject the second reagent into the cell.

15. The method of claim 13, further comprising:
monitoring if the reagent is injected into the cell; and
repeating the activating until the reagent is injected into the cell if the injection of the reagent fails based on the monitoring.
